(19) United States
(12) Patent Application Publication
(10) Pub. No.: US 2009/0274780 A1
(43) Pub. Date: Nov. 5, 2009

(54) MENOPAUSE TREATMENT COMPOSITION AND REGIMEN

(76) Inventor: Adam A. OFER, Weston, CT (US)

Correspondence Address: THADDIUS J. CARVIS, 102 NORTH KING STREET, LEESBURG, VA, 20176, US

(21) Appl. No.: 12/434,847

(22) Filed: May 4, 2009

Related U.S. Application Data
(60) Provisional application No. 61/050,234, filed on May 4, 2008.

(51) Int. Cl. A61K 36/258

(52) U.S. Cl. 424/728

(57) ABSTRACT

Disclosed are compositions and methods for treating symptoms associated with menopause and/or perimenopause. The compositions are based on a combination of concentrated extracts containing black cohosh, ginseng, red clover, soy isoflavones and St. John's wort, in the form of active compounds of these extracts. The treatment regimen, entail orally administering to a human subject suffering from symptoms of menopause and/or perimenopause, an effective dosage in effective amounts of the noted composition.

### CROSS REFERENCE TO RELATED APPLICATION

This application claims priority to copending prior U.S. Provisional Patent Application Ser. No. 61/050,234 filed May 4, 2008, the disclosure of which is incorporated herein by reference in its entirety.

### FIELD OF THE INVENTION

The invention relates generally to compositions and methods for treating symptoms associated with menopause and/or perimenopause.

### BACKGROUND OF THE INVENTION

Menopause has been defined as the point in a woman's life when she has not experienced a menstrual period for a full year. Menopause marks the end of the childbearing years and is sometimes referred to as “the change of life.” For most women, menopause happens around age 50, but there is considerable variation. Perimenopause refers to a process of change that preceeds menopause.

Commonly menopause and perimenopause will cause physiological and psychological changes in an affected subject.

While menopause is a natural part of growing older, an affected woman does not require treatment unless the symptoms become unduely bothersome. This, again, varies in individuals.

Menopause is accompanied by normal changes in reproductive and hormone systems. As the subjects' follicle (egg) supply diminishes, the body begins to ovulate less often. Dramatic changes in hormone levels occur, particularly in luteinizing hormones (LH), folicle stimulating hormone (FSH), estradiol and progesterone. This causes changes in menstrual periods and other symptoms. In time, the menstrual cycle completely stops.

A number of symptoms have been associated with menopause, among which are various changes affecting vasomotor stability, urogenital health, skeletal, skin and soft tissue and psychological well being. These changes are often experienced as vasomotor symptoms that can affect many aspects of life. These changes can disrupt daily life in some women, causing them to experience aspects of vasomotor instability, including hot flashes or hot flushes, night sweats and, in a few people, cold flashes, sleep disturbances, poor quality sleep, light sleep and insomnia.

In 1991, the National Institutes of Health (NIH) launched the Women's Health Intitiative (WHI) to address the most common causes of death, disability and impaired quality of life in postmenopausal women. It was based on a set of clinical trials and an observational study, which together involved over 160,000 generally healthy postmenopausal women. The clinical trials were designed to test the effects of postmenopausal hormone therapy, diet modification, and calcium and vitamin D supplements on heart disease, fractures, and breast and colorectal cancer.

The WHI hormone trial was comprised of two studies: the estrogen-plus-progestin study of women with a uterus and the estrogen-alone study of women without a uterus. The women with a uterus were given progestin in combination with estrogen, a treatment intended to prevent endometrial cancer. In both hormone therapy studies, women were randomly assigned to either the hormone medication being studied or to placebo. Those studies were ended when risks were observed that reached statistical significance. The women in these studies are now participating in a follow-up phase, which will last until 2010. However, because of questions that existed before the studies and some raised during them have not been fully answered, many women are seeking non-hormone therapies.

In a fairly recent review published in The American Journal of Medicine, Tieraona Low Dog, MD, assesed the evidence for safety and benefit of botanical monopreparations used for relief of menopause-related symptoms. To facilitate the assessment, Dr. Dog searched the Cochrane Library and Medline databases from January 1966 to October 2004, using a detailed list of terms related to botanicals and menopausal symptoms. Under his criteria, studies were considered eligible (1) if they were controlled trials of a botanical monopreparation administered orally for a minimum of 6 weeks to perimenopausal or postmenopausal women with hot flashes and (2) if they included a placebo or comparative treatment arm. A total of nineteen studies were included, involving: black cohosh, dong quai, evening primrose seed oil, ginseng, red clover and soy. He concluded that some of the studies were encouraging, but that more research was necessary. No synergies were reported in the assessment. (The American Journal of Medicine (2005) Vol. 118 (12B), 98S-108S)

In U.S. Pat. No. 6,346,267, Fry, et al, describe a composition and method for the treatment of symptoms associated with reduced estrogen production, such as occurs in perimenopausal and menopausal women. The composition is said to contain at least one phytoestrogen containing food, herb or extract thereof, and at least four herbs or extracts which are effective at treating conventional menopausal symptoms. Very limited clinical data compared a control composition containing red clover, soy isoflavones, black cohosh and wild yam in combination, to a test composition of that invention, which contained the same combination as the control but without the red clover and with added sage, vervain, astragalus, mother wort, and chastetree berry. The second composition was considered effective, while the control was not. They concluded that “[s]urprisingly, removal of the red clover (long thought to be a highly effective phytoestrogen) and addition of various herbs used in treating the complications as well as menopausal type symptoms gave unexpected improvements in patient well-being and relief from the effects of reduced estrogen production.”

There is a current need for compositions and treatments that provide relief of vasomotor symptoms accompanying menopause, and it would be desirable to provide the treatment in the form of a novel nutritional supplement containing concentrated herbal extracts.

### SUMMARY OF THE INVENTION

The present invention provides both a composition and a treatment regimen for relief of vasomotor symptoms accompanying menopause and/or perimenopause.

In one aspect, the invention provides a composition containing a combination of concentrated extracts containing black cohosh, ginseng, red clover, soy isoflavones and St. John's wort, preferably in the form of active compounds of these extracts. The compositions preferably exhibit synergistic benefits of the combination of these herbs to relieve the symptoms associated with menopause and/or perimenopause. It is contemplated that combinations containing less than all of these components and/or including other components will also be effective.

In another aspect, the invention provides a treatment regimen, comprising: orally administering to a human subject suffering from symptoms of menopause and/or perimenopause, an effective dosage at effective intervals and in effective amounts of a composition containing a combination of concentrated extracts containing black cohosh, ginseng, red clover, soy isoflavones and St. John's wort, preferably in the form of active compounds of these extracts.

Other preferred aspects of the invention are described below.

### DESCRIPTION OF THE DRAWINGS

The invention will be better understoon from the following detailed description, especially when read in light of the accompanying figures, wherein:

FIG. 1 is a summary of data obtained from a double-blind trial of a preferred composition according to the invention.

### DETAILED DESCRIPTION OF THE PRESENT INVENTION

The invention will be illustrated in the context of a preferred composition and a treatment regimen employing it. The composition contains a combination of concentrated extracts from black cohosh, ginseng, red clover, soy isoflavones and St. John's wort, preferably prepared to contain high concentrations of active compounds. The compositions preferably exhibit synergistic benefits of the combination of these active ingredients to relieve the symptoms associated with menopause and/or perimenopause. It is contemplated that combinations containing less than all of these components and/or including other components will also be effective.

In another aspect, the invention provides a treatment regimen, comprising: orally administering to a human subject suffering from symptoms of menopause and/or perimenopause, an effective dosage at effective intervals and in effective amounts of a composition containing a combination of concentrated extracts containing black cohosh, ginseng, red clover, soy isoflavones and St. John's wort, preferably in the form of active compounds of these extracts. The administration of these compositions preferably provides synergistic benefits of the combination of these herbs to relieve the symptoms associated with menopause and/or perimenopause. It is contemplated that combinations containing less than all of these components and/or including other components will also be effective.

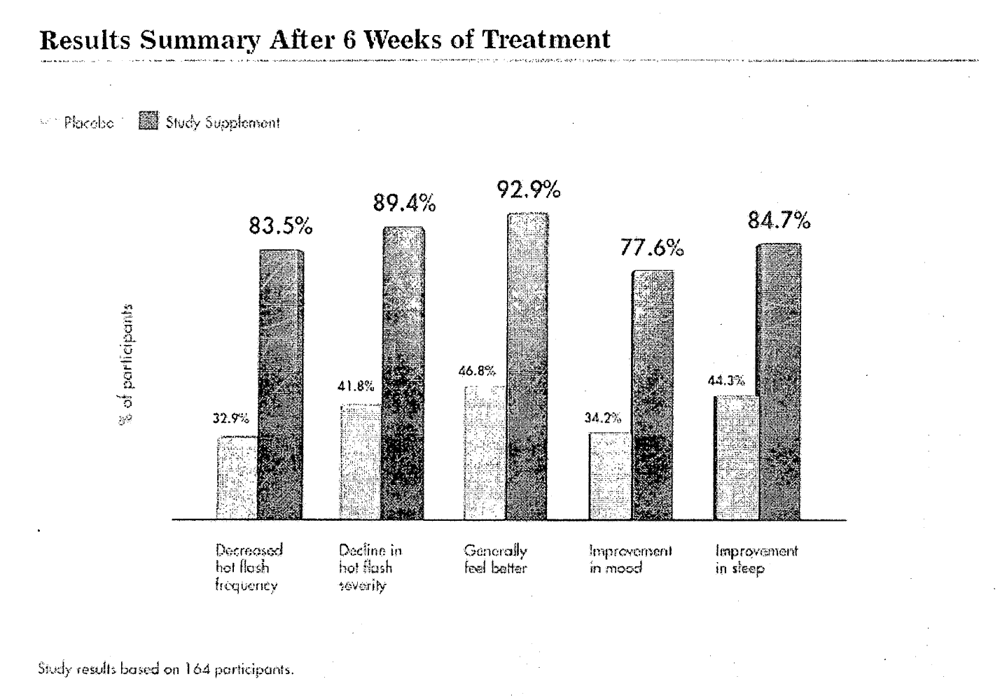

In describing the present invention, reference is made to the drawings, wherein there is seen in FIG. 1, which summarizes data from a double blind, randomized, placebo-controlled study of a preferred composition according to the invention. Patients treated with the preferred composition consistently for six weeks showed a statistically significant difference in both the frequency and severity of hot flashes and night sweats. In addition, 5 out of 6 patients reported decrease in both hot flash frequency and severity; and 3 out of 4 patients reported improvement in their mood. Participants in the study group also reported less trouble staying asleep and more than 9 out of 10 patients reported that they “generally feel better” after 6 weeks of treatment. There was no difference in side effects reported by study group versus placebo.

The compositions of the invention will contain a combination of concentrated extracts from black cohosh, ginseng, red clover, soy isoflavones and St. John's wort, preferably concentrated to contain high concentrations of active compounds. Extracts are selected from the group described and will contain compositions with activity to provide a therapeutically significant reduction of at least one symptom of menopause and/or perimenopause.

Black cohosh refers to the root and/or rhizome of black cohosh, which is native to North America (such as Remifemin). A number of commercial preparations are available. The preferred compositions will be characterized by a content of at least about 5 to 15%, more narrowly about 7 to 9%, e.g., about 8%, triterpenes. The daily dose will preferably be about 50 mg of such extracts, more broadly from about 10 to about 100 mg, or from about 30 to about 75 mg. The compositions are provided in several doses per day, e.g., about two doses per day spaced evenly time wise between morning and evening. The dosage form can be gel capsules, enteric coated for time release or a popular form such as liqui-gel, tablet, or the like. While ranges of percentages of active ingredients are given below, these are based on practical considerations such as commercial availability of quality extracts, and there is no restriction on level at the upper end of the range.

St. John's wort (Hypericum perforatum) is a shrubby perennial plant with numerous bright yellow flowers, which is native to North America. A number of commercial preparations of the St. John's wort extract are available. The preferred compositions will be characterized by a content of at least about 0.3% of hypericin, more broadly 5 to 15%, more narrowly about 7 to 9%. The daily dose will preferably be about 900 mg of such extracts, more broadly from about 100 to about 1500 mg, or from about 500 to about 1200 mg.

Another component is an extract of red clover (Trifolium pratense L.). Red clover includes red clover blossoms, which have been used in traditional herbal medicine for centuries, but for the compositions of the invention the extracts are isoflavone rich leaf extracts. A number of commercial preparations of the red clover extract are available (such as Promensil). The preferred compositions will be characterized by a content of at least about 16% Isoflavones, more broadly 5 to 25%, more narrowly about 10 to 20%. The daily dose will preferably be about 376 mg of such extracts, more broadly from about 50 to about 1000 mg, or from about 200 to about 600 mg.

Ginseng (Panax ginseng) extracts are present in the preferred compositions of the invention. A number of commercial preparations of the ginseng extract are available. The preferred compositions will be characterized by a content of at least about 80% ginsenosides, more broadly 25 to 99%, more narrowly about 60 to 90%. The daily dose will preferably be about 10 mg of such extracts, more broadly from about 2 to about 25 mg, or from about 5 to about 20 mg.

Soy extracts rich in isoflavones (Glycine max L.) are also preferred in the compositions of the invention. A number of commercial preparations of the soy isoflavones are available. The preferred compositions will be characterized by a content of at least about 40% isoflavones, more broadly 10 to 90%, more narrowly about 20 to 60%. The daily dose will preferably be about 312 mg of such extracts, more broadly from about 100 to about 1000 mg, or from about 200 to about 500 mg.

In another aspect, the invention provides a treatment regimen, comprising: orally administering to a female human subject suffering from symptoms of menopause and/or perimenopause, an effective dosage in effective amounts of a composition containing a combination of concentrated extracts containing black cohosh, ginseng, red clover, soy isoflavones and St. John's wort, preferably in the form of active compounds of these extracts.

The administration of these compositions preferably provides synergistic benefits of the combination of these herbs to relieve symptoms associated with menopause and/or perimenopause, specifically reducing the frequency and severity of one or more of the above symptoms, namely: hot flashes, night sweats, interrupted sleep and lack of generally feeling well. It is contemplated that combinations containing less than all of these components and/or including other components will also be effective.

The compositions are provided in several doses per day, e.g., up to six, but preferably about two equal doses per day spaced evenly time wise between morning and evening. The effective doses will contain within from about 25 to about 500%, more narrowly from about 50 to about 200%, and preferably from about 75 to about 125% of the amounts exemplified below.

One exemplary dosage form is shown in the examples below, which is presented to better illustrate and explain the invention and is not to be taken as limiting in any regard.

### Example 1

An exemplary, preferred composition of the invention is prepared by blending the following ingredients in the amounts indicated in the table:

| Ingredient | Amount |
|---|---|
| St. John's Wort Extract, 0.3% Hypericin | 450 mg |
| Red Clover Extract, 16% Isoflavones | 188 mg |
| Soy Extract, 40% Isoflavones | 156 mg |
| Black Cohosh Extract, 8% Triterpenes | 25 mg |
| Panax Ginseng Extract, 80% Ginsenosides | 10 mg |

These ingredients are blended with microcrystalline cellulose, magnesium stearate, silica, and titanium dioxide, and the resulting mixture is filled into gelatin capsules, preferably with the amount split between two capsules.

In a preferred treatment regimen, two capsules as thus prepared are taken orally at two times during the day, preferably morning and evening.

### Example 2

A clinical test was designed and carried out over an extended time period to determine the safety and efficacy of the novel nutritional supplement containing concentrated herbal extracts as prepared according to Example 1, for the relief of vasomotor symptoms related to menopause.

The study was a double-blind, randomized, placebo-controlled study. One hundred and eighty six (186) women with irregular or absent menstrual cycles suffering from at least five bothersome hot flashes or night sweats per 24-hour period were randomized to receive the study supplement or placebo. Study supplement consisted of capsules as prepared in Example 1, containing the standardized concentrated extracts, which provided a daily serving (split into two equal doses of two capsules each) with St. John's wort extract delivering 2.7 mg hypericin daily, soy extract delivering 125 mg isoflavones daily, red clover extract delivering 60 mg triterpenes daily, black cohosh extract delivering 4 mg triterpenes daily, and panax ginseng extract delivering 16 mg ginsenosides daily. Patients filled out a 3-day symptom diary prior to acceptance into the study and again after six weeks of study participation.

One hundred and sixty four (164) patients completed 6 weeks of study participation and returned their 3-day symptom diary. When compared to placebo, the supplement group showed a statistically significant difference in patients reporting decrease in both frequency (83.5% vs. 32.9%) and severity (89.4% vs. 41.8%) of hot flashes and night sweats. Participants in the study group also reported less trouble staying asleep and improvement in mood. There was no difference in side-effects reported by study group versus placebo. The results are summarized in FIG. 1.

From this I conclude that the use of the combination of concentrated standardized extracts in this study offers a safe and effective treatment for vasomotor symptoms associated with the hormonal fluctuations of perimenopause/menopause. It also appears effective for improvement of mood and sleep dysfunction which are often present during the hormonal transitions of menopause.

While the present invention has been disclosed and described with reference to a single embodiment thereof, it will be apparent, as noted above that variations and modifications may be made therein. It is also noted that the present invention is independent of the machine being controlled, and is not limited to the control of inserting machines. It is, thus, intended in the following claims to cover each variation and modification that falls within the true spirit and scope of the present invention.

1. A composition containing a combination of concentrated extracts containing black cohosh, ginseng, red clover, soy isoflavones and St. John's wort, in the form of active compounds of these extracts.

2. A composition according to claim 1, wherein the black cohosh is present as an extract characterized by a triterpene content of 5 to 15%.

3. A composition according to claim 1, wherein the Ginseng is present as an extract are characterized by a ginsenoside content of 25 to 99%.

4. A composition according to claim 1, wherein the red clover composition is present as an extract characterized by an isoflavones content of 5 to 25%.

5. A composition according to claim 1, wherein the soy isoflavones are present as an extract characterized by an isoflavones content of 10 to 90%.

6. A composition according to claim 1, wherein the St. John's wort is characterized by a hypericin content of 5 to 15%.

7. A composition according to claim 1, comprising black cohosh extract characterized by 5 to 15% triterpenes, ginseng extract characterized by a ginsenoside content of 25 to 99%, red clover extract characterized by an isoflavone content of 5 to 25%, soy extract characterized by an isoflavone content of 10 to 90% and St. John's wort characterized by a hypericin content of 5 to 15%.

8. A daily dosage of a composition according to claim 1, comprising in at least two capsules, from 25 to 500% of the amounts of the ingredients as follows:

| Ingredient | Amount |
|---|---|
| St. John's Wort Extract, 0.3% Hypericin | 450 mg |
| Red Clover Extract, 16% Isoflavones | 188 mg |
| Soy Extract, 40% Isoflavones | 156 mg |
| Black Cohosh Extract, 8% Triterpenes | 25 mg |
| Panax Ginseng Extract, 80% Ginsenosides | 10 mg |

9. A daily dosage of a composition according to claim 8, comprising from 75 to 125% of the amounts of the ingredients shown in claim 8.

10. A treatment regimen, comprising: orally administering to a human subject suffering from symptoms of menopause and/or perimenopause, an effective dosage at effective intervals and in effective amounts of a composition containing a combination of concentrated extracts containing black cohosh, ginseng, red clover, soy isoflavones and St. John's wort, in the form of active compounds of these extracts.

11. A regimen according to claim 10, wherein a daily dose will comprise from 10 to 100 mg of black colish extract, from 2 to 25 mg of ginseng extract, from 50 to 1000 mg of red clover extract, from 100 to about 1000 mg of soy extract and from 100 to 1500 mg St. John's wort.

12. A regimen according to claim 10, wherein the composition comprises black cohosh extract characterized by 5 to 15% triterpenes, ginseng extract characterized by a ginsenoside content of 25 to 99%, red clover extract characterized by an isoflavone content of 5 to 25%, soy extract characterized by an isoflavone content of 10 to 90% and St. John's wort characterized by a hypericin content of 5 to 15%.

13. A regimen according to claim 12, wherein a daily dose will comprise from 10 to 100 mg of black colish extract, from 2 to 25 mg of ginseng extract, from 50 to 1000 mg of red clover extract, from 100 to about 1000 mg of soy extract and from 100 to 1500 mg St. John's wort.

14. A regimen according to claim 10, wherein the composition is administered in equal doses up to six times a day.

15. A regimen according to claim 13, wherein the composition is administered in equal doses two times a day.
